(19) United States
(12) Patent Application Publication
(10) Pub. No.: US 2004/0096817 A1
(43) Pub. Date: May 20, 2004

(54) Model for in vitro adhesion development

(75) Inventors: Michael P. Diamond, Grosse Pointe Shores, MI (US); Ghassan M. Saed, West Bloomfield, MI (US)

Correspondence Address: ROPES & GRAY LLP, ONE INTERNATIONAL PLACE, BOSTON, MA, 02110-2624, US

(73) Assignee: Wayne State University

(21) Appl. No.: 10/447,454

(22) Filed: May 28, 2003

Related U.S. Application Data
(60) Provisional application No. 60/385,160, filed on May 31, 2002.

(51) Int. Cl. C12Q 1/00; C12N 5/08

(52) U.S. Cl. 435/4; 435/366

(57) ABSTRACT

A biological model for the development of adhesions in vitro comprises a pair of opposed surfaces of tissue explants maintained in a culture media for a sufficient time and under conditions to permit the formation of adhesions. The model is useful for evaluating compounds and techniques for the prevention and remediation of adhesions, and for individualizing the therapeutic options for patients who may experience adhesions.

### CROSS-REFERENCE TO RELATED APPLICATION

This application claims the benefit of co-pending disclosure of provisional application No. 60/385,160, filed May 31, 2002, which is incorporated by reference herein in its entirety.

### BACKGROUND OF THE INVENTION

The postoperative development of adhesions complicates the healing process in the overwhelming majority of men and women who undergo intraabdominal surgery. Adhesions are scar tissue that connects other tissue in anatomically abnormal locations. These adhesions are a major contributing factor to infertility, abdominal and pelvic pain, and bowel obstruction, and they complicate reoperative procedures. Thus, intraabdominal adhesions are an important medical problem, because they are a source of significant patient morbidity and create the potential for intraoperative/post-operative complications, including even mortality. In addition, adhesions constitute an important economic burden, costing patients and third-party carriers over one billion dollars a year.

The development of adhesions is, in essence, an abnormal manifestation of the healing process. However, it is not understood why adhesions differ in severity among patients, nor is it understood why they develop at one location but not in another within the same patient. Most importantly, it is not known how the adhesion formation process can be manipulated to minimize or eliminate the development of adhesions postoperatively. Much of the reason for these gaps in knowledge stem from the lack of a system in which the process of adhesion development can be suitably studied and modified.

The conduct of such studies has been limited, because clinical conditions that would ethically allow a second look are also limited, due to the potential risks to which patients would be exposed. Animal models of adhesion development have generally been unsatisfactory, because outcomes have not been reproducible, especially in the hands of different surgeons. Furthermore, the results obtained in experimental animals are not necessarily representative of the processes in human patients. The creation of an in vitro model that faithfully mimics the process of adhesion development in humans would be beneficial by allowing its manipulation, thereby allowing the development of an understanding of how the adhesion formation process can be interdicted.

Surgeons and their patients are faced daily with the problem of intraabdominal adhesions and the complications that such adhesions create. Because of a limited knowledge of how the process of adhesion formation can be manipulated, physicians have been unable to provide suitable levels of care that either reduces or prevents the development of adhesions.

Postoperative adhesions occur in the overwhelming majority of patients after laparotomy and laparoscopy. The failure by clinicians to identify and acknowledge this well-established fact is due, in large part, to the ethical and logistical restraints on their abilities to perform routine second-look operations, at which time they could assess the occurrence of postoperative adhesions. It has been found that approximately 85% of patients undergoing intraabdominal surgeries develop adhesions. Postsurgically, adhesions develop within days of the procedure. After a few days, adhesions do not change in incidence or extent of involvement at these sites. However the severity of adhesions worsens over time, with adhesions less likely to be filmy and avascular and more likely to become dense and/or vascular. While most initial studies have been conducted using infertility patients, a recent report has extended these observations to women without infertility and to men. For example, at second-look, 94% of males and females who had undergone a colectomy had adhesions immediately beneath the midline abdominal incision. Becker, J M et al., Journal American College of Surgery, 183, pages 297-306 (1996).

The personal health consequences of adhesions can be devastating. Adhesions are estimated to contribute to infertility in 40% of infertile couples, and they are the most common cause of small bowel obstruction, which can occur either in the immediate postoperative period or as late as more than twenty years following surgery. Although controversial, the presence of abdominal-pelvic pain has also often been attributed to adhesions: adhesions are estimated to be a contributing factor in up to 30% of women with chronic pain. El-Mowafi, DM et al., Surgical Technology, pages 273-283 (1998). Even for a patient who does not suffer a major complication, at the time of a later surgical procedure, it takes longer to enter the individual's abdomen, and there is increased risk of intraoperative injuries to the bowel, bladder, blood vessels or other sites. Finally, prior scarring associated with excessive intraperitoneal tissue repair can lead to serious complications for peritoneal dialysis patients, or those who must receive drugs or nutrition by the intraperitoneal route.

In spite of these many reasons to address the problem of adhesions, and despite the large number of surgical procedures performed each day in this country, the understanding of peritoneal healing is extremely limited. One thing stands out, however: some patients heal adhesion-free, while others develop severe scarring from seemingly equivalent procedures. The basis for this predisposition is completely unknown. Similarly, in a single patient, it is unclear why postoperative adhesions develop at one surgical site and not another. There is a current effort underway by researchers to identify patterns of molecular markers associated with in vivo adhesion development.

The clinical significance of the consequences of adhesions is further highlighted by a recent report that among individuals who had their first intraabdominal procedure performed in 1986, approximately 35% had at least one or more hospitalizations for adhesions over the next ten years; 5.7% had findings definitely attributable to adhesions, while an additional 22% had findings possibly consistent with adhesions. Ellis, H et al., Adhesion-related Hospital Readmissions after Abdominal and Pelvic Surgery: A Retrospective Cohort Study. Additionally, the clinical consequences of adhesions result in a huge economic burden, having been estimated in the United States when considering adhesiolysis procedures alone to cost 1.18 billion dollars in 1988 and 1.3 billion dollars in 1994, even when excluding economic consequences from time lost from work. Thus, because of the large numbers of patients in need of intraabdominal surgeries in hospitals throughout the country each day in which adhesions may be formed, the clinical consequences which adhesions have, and the cost to society from their treatment, the evaluation of the pathogenesis of adhesions is of high importance.

Accordingly, it is an object of this invention to provide an in vitro model of adhesion development that will facilitate the understanding of the adhesion formation and development process at the molecular level, and would lead to the vertical advancement of related surgical and basic sciences.

Another object is to enhance the ability to study how this process can be manipulated and suppressed in vitro, thus leading to novel approaches that would either minimize or eliminate the development of adhesion-related postsurgical complications, leading to improved patient outcomes, and reducing the cost of dealing with adhesion-related postsurgical complications.

A further object is to utilize the knowledge gained from studying the mechanism of adhesion development to provide new, targeted therapeutic approaches to reduce adhesions in patients, thereby greatly minimizing the many adverse consequences and complications they cause, and improving the health and quality of life of patients.

An additional object is to utilize the model as a screening tool for the identification of small molecules or devices, such as barrier materials, or treatment modalities. A still further object is to identify the optimal treatment agents to be used in conjunction with specific individuals, and to develop kits incorporating tissue samples from individual patients.

### SUMMARY OF THE INVENTION

The objectives of this invention are addressed by an in vitro model of adhesion development that can be used to evaluate therapies for treating humans at risk of developing adhesions. Prior to the present invention, the direct study and manipulation of the process of adhesion development in humans was problematic. This is a result, in part, of the lack of suitable imaging modalities to identify adhesions in patients, and the lack of suitable serum markers or other methods to detect the development of adhesions. Visualization at the time of a second surgical procedure has heretofore been the only approach available.

According to the present invention, the process of adhesion development is assumed to result from a combination of tissue injury, local hypoxia, and the presence of a fibrinous exudate that subsequently becomes organized into mature scar tissue (the adhesion). In the in vitro adhesion model, tissue samples from a mammal, preferably a human, are used to evaluate the effectiveness of a particular treatment modality. Suitable tissues include peritoneum tissue, visceral tissue, subcutaneous tissue, and skin tissue. The modalities to be evaluated can include any of a variety of therapeutic drugs, barrier materials, compounds, molecules, biological substances, antibodies, antisense DNA, and instruments or devices. The tissue surfaces are spaced apart 1 mm to 20 mm, and preferably 3 mm to 7 mm.

Explants of traumatized or untraumatized parietal peritoneum are cultured and positioned adjacent and facing one another for a period of, for instance, up to five days, in the presence or absence of a fibrin clot, in a chamber containing culture medium and from 0% to 21% oxygen. The tissue explants can be advantageously embedded in a material selected from the group consisting of collagen, synthetic extracellular matrix material, and natural extracellular matrix material. The culture medium can include an exogenous pro-fibrotic substance, such as TGF-β1 or talc. The effectiveness of a particular treatment modality is determined by the extent of adhesion formation in the in vitro adhesion model.

The present invention also includes kits specific for individual patients. The kits contain tissue samples from the patient, and are used to develop individualized therapeutic approaches for that patient to minimize adhesions by, for instance, screening the available therapies against the specific tissue sample, and selecting the therapy most appropriate for that patient.

### DETAILED DESCRIPTION OF THE INVENTION

The peritoneum and the serosal surfaces of organs within the peritoneal cavity are composed of mesothelial cells and sub-mesothelial tissue that contain fibroblasts, macrophages and blood vessels. Injuries to these surfaces, either due to infection, pathologic processes, and/or tissue trauma induced during surgical procedures, result in the development of adhesions in the vast majority of patients. The biological processes that result in either uncomplicated repair or the development of adhesions include migration, proliferation, and/or differentiation of several cell types, including inflammatory and immune cells, mesothelial cells and fibroblasts. Substances produced locally by these cells regulate fibrinolytic activity, tissue remodeling, angiogenesis, synthesis, and the deposition of ECM, and are central to the development of adhesions. Orchestration of this process is such that it is complete in three to five days, by which time remesothelialization of the peritoneal surface will occur. Thus, this short time span is the critical period during which adhesions form, or the surfaces heal adhesion free.

TGF-β plays a key role in the adhesion formation processes as a result of its activities which include the recruitment of inflammatory cells and fibroblasts; the regulation of synthesis, deposition and turnover of extracellular matrix components; and the modulation of angiogenesis, as well as the stimulation of tissue fibrosis at sites throughout the body. TGF-β1 has been identified in animals at sites of peritoneal trauma, with higher levels in injured as opposed to uninjured areas. Higher levels have been found in peritoneum in humans with adhesions as opposed to peritoneum without adhesions.

One of the potential early consequences of peritoneal injury is the reduction of fibrinolytic activity, which can result in the persistence of the provisional matrix, which is a fibrin-rich exudates that results from peritoneal injury. Removal of this matrix requires the activity of plasmin, a serine protease. Plasmin activity represents a balance between plasminogen activator activity (particularly tPA) and plasminogen activator inhibitors (particularly plasminogen activator inhibitor 1 or PAI-1). TGF-β reduces plasminogen activator synthesis, while increasing PAI-1 expression. Plasminogen activator activity is reduced at sites of surgical trauma, as well as in individuals who develop adhesions, primarily because of increased PAI-1 expression, while administration of tPA reduces adhesion development. Furthermore, in patients with adhesions, tPA activity is reduced in adhesions as compared to the adjacent peritoneum.

Many components of the extracellular matrix have been identified in the wound healing process. The overexpression of these components results in tissue fibrosis. In particular, collagen and fibronectin play a key role in cell growth, differentiation, angiogenesis and cell migration. The extracellular matrix also plays a crucial role in cell-to-cell interaction and communication which occurs in part through membrane bound integrins, as well as in serving as a binding site for cytokines and growth factors, thereby providing a method to allow their sequestration, and providing a mechanism to regulate their availability at the cell surface. TGF-β can stimulate the expression of collagen and fibronectin by fibroblasts; collagen III and fibronectin are deposited in the initial phase of wound healing, with collagen III subsequently gradually replaced by collagen I. Mesothelial cells have been shown to express both collagen I and III and fibronectin. Furthermore, TGF-β1 and hypoxia have been shown to regulate the expression of collagen type I and III in human peritoneal mesothelial cells. Hypoxia has been shown to result in a 1.9-fold increase in TGF-β1 mRNA expression compared with controls. The ratio of collagen type III to type I also increases under both hypoxic conditions and TGF-β1 treatment. This suggests that hypoxic conditions modulate ECM production in mesothelial cells through a mechanism involving TGF-β1 expression.

Matrix metalloproteinases (MMPs) are a family of zinc proteases that hydrolyze various components of the extracellular matrix, such as collagens, fibronectin, elastin, and proteoglycans. The MMPs are divided into subgroups by their substrate specificities and include collagenases (MMP-1, MMP-8, MMP-13), gelatinases (MMP-2, MMP-9), stromelysins (MMP-3, MMP-7, MMP-10, MMP-11), matrilysin (MMP-9), and the newly discovered membrane-type MMPs (MT-MMP). MMP's are produced as inactive proenzymes; activity requires activation that can be achieved by factors including serine proteases such as plasmin and trypsin. Activity of MMPs is regulated, at least in part, by tissue inhibitors of metalloproteinases (TIMPs), of which four have been identified. TIMP-1 and TIMP-2 inhibit all MMPs by forming a complex in a 1:1 ratio. MMPs are usually expressed at low levels in adult tissues unless induced by tissue injury, while TIMPs are usually expressed at high levels in tissues and are regulated in coordination with MMPs.

TGF-β1 has been shown to decrease the expression of MMPs while increasing TIMPs, thereby decreasing matrix degradation and increasing tissue fibrosis in cutaneous wound healing. Peritoneum and adhesion tissue express MMPs and TIMPs mRNA and protein; TIMP-1 is significantly higher in adhesions than in peritoneum and parallels the increase in TGF-β1 expression. Furthermore, in peritoneal mesothelial cells and adhesion fibroblasts in culture, TGF-β1 increases the levels of collagen I, collagen III, fibronectin and TIMP-1, while reducing the expression of MMP-1, MMP-3, and mRNA. These effects are time-dependent, occurring maximally after 6 to 12 hrs of treatment. At the protein level, TGF-β1 has been shown to increase the release of MMP-1 and TIMP-1 by mesothelial cells during the early treatment period with peak values occurring after 12-24 hrs of treatment, compared to controls.

Tissue oxygen is also an important factor in wound healing. Devascularization directly slows the healing of granulating wounds, while low tissue oxygen tension has been associated with an increased leakage rate following colonic anastamosis. Furthermore, varying the oxygen tension influences the rate of healing of open wounds, being accelerated by hyperoxia and impeded by hypoxia. In part, this may reflect the changes in the healing process that are associated with low tissue oxygen levels, and can result in adhesion development as a compensatory mechanism within the body in an attempt to resupply oxygen and nutrients to hypoxic tissues.

Hypoxia also induces alterations in multiple factors having a critical role in adhesion development. For example, in cultures of human mesothelial cells, twenty-four hours of hypoxic treatment (2% O₂) in culture increases TGF-β1 mRNA expression nearly two-fold. Additionally, hypoxia increases the expression of collagen III but not collagen I mRNA. This is consistent with 2% oxygen stimulation of TGF-β1 and procollagen syntheses. Similarly, in cultures of fibroblasts from normal human peritoneum, hypoxia increases TGF-β1, TGF-β2, MMP-1, and TIMP-1, with more profound increases from fibroblasts derived from adhesions for TGF-β1, TGF-β2, IL-10, TIMP-1, type I collagen, and fibronectin. MMP-1 expression by adhesion fibroblasts also decreases when exposed to hypoxia. Finally, VEGF is now thought to be a mediator of hypoxia-induced angiogenesis, an effect that is thought in turn to be mediated by hypoxia-inducible factor-1 (HIF-1).

The in vitro adhesion model of the present invention comprises at least two opposed, viable explants of mammalian, and preferably human, tissue maintained in culture under conditions sufficient to permit the formation of adhesions on one or more of the explant surfaces. Suitable tissue types for use in the invention include peritoneum tissue, visceral tissue, subcutaneous tissue and skin tissue. The tissue can be traumatized or non-traumatized, and a blood clot can be inserted or formed between the tissue to accelerate the production of adhesions. The culture medium for the adhesion model can include oxygen at a concentration of from about 0% to about 21% oxygen by volume, preferably less than 5% oxygen by volume. The culture medium can also include exogenous pro-fibrotic agents, such as TGF-β1 or talc.

Typically, the opposed tissue surfaces are separated by about 1 mm to about 20 mm, and preferably by about 3 mm and 7 mm. By “opposed”, in the context of this invention, is generally meant that the tissue surfaces are spaced apart and maintained in a roughly parallel relationship with each other, with the spacing gap between the tissues generally being in the range of 1 mm to 20 mm. The tissue explants can be advantageously embedded in a material selected from the group consisting of collagen, synthetic extracellular matrix material and natural extracellular matrix material.

Formation of an adhesion in the in vitro model can occur by maintaining the tissue explants in culture for a time period of from about 2 to about 5 days. The extent and speed of adhesion formation will vary depending on the composition of the culture medium, and other parameters of the medium such as temperature, pH, etc. Various exogenous agents can be added to the medium to accelerate or retard the formation of adhesions. Adhesion formation in the presence of a therapeutic formulation, a barrier material, an instrument or a device can be evaluated by comparing the speed and extent of adhesion formation with a suitable baseline or control. In this manner, a treatment applicable to a broad population of patients can be developed, or a therapeutic regime targeted to a particular individual patient can be suitably optimized.

A in vitro adhesion model of this invention can also be incorporated in a kit with other kit components, such as anti-adhesion agents, for use in diagnosis and developing a treatment for a specific patient.

The morphogenic characteristics of the in vitro model in relation to human adhesion development can be assessed using H and E staining, and special staining for collagen, to determine the extent to which in vitro processes morphologically mimic the in vivo processes. The extent to which the model mimics the in vivo molecular process of adhesion development in human beings is monitored using changes in markers such as transforming growth factor-beta (TGF-β), matrix metalloproteinases (MMPs), tissue inhibitors of matrix metalloproteinases (TIMPs), plasminogen activator (PA), plasminogen activator inhibitor-I (PAI-1), collagen I, collagen III, and fibronectin. See, for instance, U.S. Provisional patent application No. 60/275,349 and U.S. Provisional patent application No. 60/275,381, and the corresponding U.S. regular applications, the respective disclosures of which are incorporated herein by reference in their entireties.

The following examples are illustrative of the various embodiments and aspects of the invention, and are not intended in any way to limit or restrict the scope of the invention as set forth in the appended claims.

### EXAMPLE 1

The purpose of this example is to demonstrate that viable explants of parietal peritoneum tissue can be maintained in vitro long enough to support the development of adhesions.

Segments of the parietal peritoneum are excised to create tissue explants 1 cm in length by 1 cm in height by approximately 2-3 mm in width. Harvested explants are then immediately rinsed in standard media (DMEM medium containing 10% fetal bovine serum, 2% penicillin, and streptomycin) to remove blood, and then transferred to 1 ml of the same medium in a sterile culture dish. Cultures are maintained in a humidified 37° C. incubator with 20% oxygen/80% nitrogen for a total of 5 days, with the exchange of media occurring 3-4 times each day. At the conclusion of the five days of culture, tissue viability is assessed by crystal violet and trypan blue exclusion. These results demonstrate the ability to maintain viable tissue explants throughout the five day period proposed for the in vitro model of adhesion development.

### EXAMPLE 2

The purpose of this example is to demonstrate the viability of an in vitro model of adhesion development.

It is hypothesized that postoperative adhesions develop at localized sites of tissue hypoxia at the sites of peritoneal injury in the presence of a fibrinous exudate which originates from blood or transudates from the injured peritoneum. Adhesions occur when this clot persists long enough to allow fibroblast infiltration from the underlying tissues before fibrinolysis occurs. Once infiltrated, the fibroblasts produce collagen, fibronectin, and other extracellular matrix components which become covalently cross-linked, thereby creating a band, or “an adhesion”. Over time, particularly under hypoxic conditions, angiogenesis will occur, resulting in vascularization of the adhesion. Concurrently, continued occurrence of extracellular matrix deposition can transform filmy adhesions into more dense, opaque adhesions.

Experimental Design

To test the above hypothesis, in vitro cultures of tissue explants are created to evaluate all eight combinations of the three variables of the model: 1) traumatized vs. non-traumatized peritoneum, 2) culture oxygen concentrations of 20% vs. 2% (e.g., normoxic and hypoxic oxygen concentrations), and 3) the presence or absence of a preformed blood clot between the opposing peritoneal surfaces. The in vitro adhesion model makes use of harvested tissue explants obtained from peritoneum adjacent to the anterior abdominal wall incision line. Specifically, a skin incision is made and carried down through all tissue layers to enter the abdominal cavity. After exposing the peritoneum, two segments parallel to each side of the peritoneal incision line 12 cm long by 1 cm in height by 2-3 mm in width are excised. This provides sufficient tissue for the eight 1×3 cm explants required for the eight model preparations.

For examination of the effects of tissue trauma, traumatized explants are compared with explants in which special care has been taken to insure absolutely no touching of the peritoneal surface during harvesting, preparation, and culturing. Once harvested, explants are rinsed in standard media (DMEM with 10% fetal bovine serum, 2% penicillin and streptomycin). Traumatization of the peritoneum is achieved by abrasion twenty times with a scalpel blade. Traumatized and non-traumatized explants are folded in the middle lengthwise such that the peritoneal surfaces are positioned adjacent to each other in a dish of media such that the explant is completely covered by media, and the peritoneal surfaces are opposing each other separated by approximately 5 mm at the open edge of the explant. A single 2-0 Nylon suture is placed at the open edge to maintain the explant in this configuration with the peritoneal surfaces approximated. This configuration allows the potential for an “adhesion” to develop between the two peritoneal surfaces. The model preparation is cultured for up to five days in a humidified 37° C. incubator. This time period is chosen because in vivo, adhesion development, or healing without adhesions, occurs in three to five days.

To determine whether the culture oxygen concentration influences adhesion development, the traumatized explants are cultured in room air or at 2% oxygen (hypoxic conditions). This concentration is chosen because the growth of human peritoneal mesothelial cells and fibroblasts is altered at these concentrations.

Culturing is conducted in an airtight Plexiglas chamber (Bellco Glass, Vineland, N.J.). The chamber is gassed by a positive infusion of 20% oxygen/80% nitrogen, or 2% oxygen/98% nitrogen gas mixture, respectively. Culturing of all explants is conducted in a humidified tissue incubator with media changed 3-4 times per All experiments are performed in triplicate. All media are kept for protein analysis by ELISA assays. Additionally, total RNA and protein is extracted from all tissues for multiplex RT/PCR and western blot analysis, respectively, as described below. Since the explants are not perfused by blood, there is no “bleeding” from cut edges and transudation from the surface is limited. Consequently, to create an environment similar to that which occurs in vivo, a “clot” is placed between the peritoneal surfaces in half of the preperations.

The clot is produced by allowing 3 ml aliquot of autologous blood to clot for twenty minutes. The clot is positioned to cover the peritoneal surfaces, with the excess extending beyond the explant margins. This volume of blood is chosen by examining the amount generally needed to fill the space between the peritoneal surfaces. Care is taken during changing of the media to not disturb the relationship of the model preparation. Control explant cultures are conducted in an analogous manner, but without addition of clot. At the completion of culturing, gross visual examination is performed to determine whether an “adhesion” exists bridging the opposing explants. The adhesion preparation is prepared for histological or molecular examinations.

Results

Placement of the clot between opposing traumatized peritoneal surfaces under hypoxic conditions results in the development of an “adhesion”. This is expected because traumatization of the surfaces alters the microenvironment, initiating changes such as an increase in TGF-β1, TIMP-1, and PAI-1, along with reductions in MMP-1 and PA, thereby creating an environment with decreased fibrinoloysis and tissue remodeling.

The model presented here, opposing traumatized peritoneal surfaces separated by a blood clot, is intended to mimic what occurs following intraabdominal surgeries in men and women, which leads to adhesion development in vivo. If a n adhesion fails to develop, the examination of plasminogen activator activity in the explants can be conducted, and if present, indicates that increased traumatization of the tissue is needed. This is not expected, because abrasion with the scalpel disrupts the mesothelial surface. If necessary, in order to increase the stimulus for adhesion development, exogenous administration of TGF-β1, or talc, or other pro-fibrotic agents, can be added. Exogenous administration of these components simulates the in vivo promotion of adhesions These components can be added to the clot and/or to the incubation media. Another alternative is to embed the explants in collagen and/or the extracellular matrix (ECM) to provide a matrix within which the adhesions can develop. This has the advantage of providing a network for fibroblast in growth. However, the network would also be present at sites with high plasminogen activator activity, thereby making it less similar to the milieu in which adhesions develop in vivo.

### EXAMPLE 3

The purpose of this example is to assess the morphogenic characterization of the adhesion model in relation to human adhesion development.

In vivo, the development of an adhesion results in the presence of a new anatomic structure (the adhesion) bridging two opposing surfaces. The adhesion contains fibroblasts which have deposited extracellular matrix including collagen and fibronectin. A successful in vitro adhesion model should create an adhesion containing these attributes, and components of the paradigm (e.g., tissue injury, hypoxia, and/or fibrinous exudate) that are required for its development.

Experimental Design

In this example, the optimized in vitro adhesion model(s) described in Example 2 are used. Tissue is harvested at times 0, 24, 72, or 120 hours on at least three occasions each. At the completion of culturing, the tissue of the explant model is divided. One portion is prepared for standard histology by replacing the culture media with 10% paraformaldehyde for twenty-four hours. This specimen is then embedded in paraffin and sectioned to allow for standard H and E staining for fibroblast infiltration of the clot, as well as specialized staining for collagen deposition bridging the clot, thereby connecting the explants. The model can be considered to be successful if the histologic appearance of the in vitro adhesion bridging the gap between the explants is consistent with the appearance of adhesions identified in vivo.

Results

The use of non-traumatized peritoneum in this model results in the failure of adhesion development, because 1) plasminogen activator activity is maintained, thereby resulting in the resolution of the blood clot, and 2) the fibroblasts do not migrate through the intact mesothelium of the peritoneum. If an adhesion does develop, it could be the result of injury to the cut edges of the peritoneum sufficient to suppress plasminogen activator activity, or because the mesothelial lining of the explants is injured, thereby allowing fibroblast migration. These possibilities can be tested by hisotologic examination of the peritoneum for determination of whether the mesothelium is intact. Plasminogen activator and PAI-I mRNA and protein levels can be localized throughout the explants by in situ hybridization and immunohistochemistry, respectively.

The tissue oxygenation affects adhesion development in this explant model. Specifically, culturing under hypoxic conditions increases the magnitude of the adhesion between the opposing explant surfaces, and to further accentuate the anticipated molecular changes, namely reductions in MMP-1, MMP-3, and PA, and increases in TGF-β1, VEGF, PAI-1, TIMP-1, TIMP-2, collagens I and III, and fibronectin. These variations are enhanced when the tissue has been exposed to hypoxia. Additionally, tissue perfusion under hypoxic conditions induces expression of oxygen sensitive molecules such as HIF-1, erythropoietin, transferrin, the transferrin receptor, VEGF, nuclear factor-κβ (NF-κβ), glucose transporters, and enzymes of the glycolytic pathway. Failure to observe these changes may occur if the atmosphere culture oxygen levels (2% vs. 20%) fails to alter media, or particularly tissue, oxygen concentrations. To test this possibility, the media and tissue oxygen levels are measured using the model 210 INSTECH Fiber Optic Oxygen Monitor. If differences in tissue oxygen levels are not observed at the proposed gas oxygen percentages, the oxygen percentage can be altered to achieve variations in explant oxygen levels during culture.

### EXAMPLE 4

The purpose of this example is to assess the extent to which the adhesion model faithfully mimics the molecular process of adhesion development in humans.

The milieu at sites where adhesion develop has been characterized by specific, integrated changes including reductions in plasminogen activator and metalloproteinase activity, and increases in the levels of TGF-β1, collagen I and III, and fibronectin. The reduction in plasminogen activator and metalloprotease activity are due to both reductions in levels of PA, MMP 1, and 3 respectively, as well as to increased levels and production of PAI-1 and TIMPs 1 and 2. In this example, we determine whether the peritoneum explants and adhesions manifest these molecular changes as compared to the control explant cultures without the clotted blood.

Experimental Design

The optimized version of the model identified above is utilized in this example. Tissues are harvested from the adhesion model at times 0, 24, 72, or 120 hours on at least three occasions each. Comparison is made of gene expression at both mRNA and protein levels for each of the selected molecules in explants prepared under the eight experimental paradigms without adhesions. Enzyme activity is determined by zymography. Other tissue portions are immediately rinsed with ice-cold physiological saline (PBS) several times to remove the blood, cut into several pieces, and processed for availability for future in situ hybridization and immunohistochemical studies for each molecule.

A. Laboratory Techniques

1. Tissue Homogenization

Frozen tissues collected after culture are homogenized to a fine powder with a mortar and pestle under liquid nitrogen as previously described. Clots and/or bands of adhesions between tissue specimens are collected and processed separately from the adjacent tissue specimens. Aliquots of tissues (1 g) are mixed with the Trizol reagents and further homogenized for 60 seconds with a polytron (Brinkmann Instruments, Inc., Westbury, N.Y.) before proceeding to RNA isolation. In our hands, 35 mg of tissue yields approximately 60 μg total RNA.

2. Gene Transcription Assays

a. Multiplex RT/PCR

The multiplex RT/PCR technique has been developed to quantitate mRNA levels of key molecules that play an important part in the wound healing process. PCR primers are designed that can amplify β-actin and the molecule of interest in the same sample simultaneously. This can be done individually for TGF-β, type I collagen, type III collagen, fibronectin, VEGF, PA, PAI-1, MMP-1, and TIMP-1.

RT/PCR: Total RNA is isolated using the monophasic solution of phenol and GITC/Trizol method as described in Saed, G M et al., Biochem. Biophys Res. Comm., 203, pages 935-942 (1994).

Removal of DNA contamination from RNA: Total RNA is treated with DNase 1 RNase-free in 10 mM Tris-Cl, pH 8.3, 50 mM KCL, 1.5 mM MgCl₂ in the presence of RNase in ribonuclease inhibitor, and incubated at 37° C. for 30 min. After extraction with phenol/chloroform and ethanol precipitation, the RNA is redissolved in DEPC-treated water.

cDNA synthesis: 20 g of total RNA is heated to 68° C. for 10 min in the presence of 2:1 oligo dT primer, and then rapidly chilled on ice. A master mix containing 4:1 5× first strand buffer, 2:1 0.1 M dithiothreitol, 1:1 10 mM dNTP mix, 1:1 superscript II (200 U; Life Technologies, Inc.), and 1:1 RNase inhibitor is added, and each reaction is incubated at 42° C. for 1 h.

PCR Amplification: Aliquots from the cDNA reaction are PCR-amplified in a 100:1 reaction as follows: 10:1 of 10×PCR reaction buffer, 1 mM from each of the deoxynucleoside triphosphates, 20 mM from each primer, and 2.5 U Taq polymerase enzyme. Mineral oil is added to prevent evaporation. The reaction is initiated by heat denaturation at 95° C. for 1 min., annealing the primers for 2 min. at 60-65° C. (depends on the primer combination), and then extension for 3 min at 72° C. This is repeated for 35-40 cycles using PCR (Perkin-Elmer). After the final cycle, the temperature is maintained at 72° C. for 7 min to allow completion of the synthesis of amplified products. Analysis of PCR-amplified products is by fractionation over a 6% polyacrylamide gel, followed by silver staining of DNA bands. Scanning densometer is used to determine the ratio of intensity of each band relative to β-actin.

Primer design and controls: Optimal oligonucleotide primer pairs for multiplex RT/PCR amplification of oligo dT-primed reverse-transcribed cDNA are selected with the aid of the computer program Oligo 4.0 (National Biosciences, Inc., Plymouth, Minn.). Sets of primers that work in multiplex with β-actin for each of the molecules tested in this study are designed, calibrated, and tested. Since quantitative application of this method is contingent upon the analysis of the PCR products during the amplification phase, prior to the plateau, cycle relationships and dilutional curves for cDNA for each molecule and the housekeeping gene P-actin are determined.

b. In Situ Hybridization

Portions of tissues are collected as described above, fixed with 2% paraformaldehyde, paraffin embedded and processed as described in Dou, Q. et al., Mol. Hum. Reprod., 3, pages 383-391 (1997) and Zho, Y. et al., Biol. Reprod., 53, pages 923930. Sections (5 μm thick) are cut, collected on poly-L-lysine coated slides, deparaffinized, rehydrated in PBS and treated with proteinase K (1.0 μg/ml). The antisense and sense oligomers, used as probes for all molecules studied, are labeled with digoxigenin or ³⁵S-labeled. The probes are dissolved in a hybridization solution, and prehybridized for 1 hr at 24° C., blotted dry and hybridized overnight at 50° C. in a humidified chamber. Slides are washed and treated with RNase A, washed and either used with an alkaline phosphatase kit or processed for autoradiography for observation, according to the protocol previously described. Detection of the digoxigenin-labeled oligomers is performed using alkaline phosphatase-conjugated anti-digoxigenin antibody diluted 1:500, and incubated for 1 hr. Colorogenic reaction is performed with 5-bromo-4-chloro-3 indolyl phosphate and nitro blue tetrazolium salt, and viewed by light microscopy. Sections hybridized with sense oligomer probes are used as controls. Autoradiography is used for the detection of ³⁵S-labeled oligomers.

3. Determination of Protein Levels and Localization

a. Western Blots

Protein levels of the proteases, extracellular matrix, and growth factors are determined using western blot analysis. This is done on total cellular protein isolated from the above tissues described in Saed, G M et al., Circulation Research, 67(2), pages 510-516 (1990). Tissues are homogenized in cell lysis buffer (50 mM Tris-HCL, pH 7.4; 1% NP-40; 0.25% sodium deoxycholate; 150 mM NaCl; 1 mM EGTA; 1 mM PMSF; 1 μg/ml aprotinin, leupeptin, pepstatin; 1 mM Na3VO4; 1 mM NaF). SDS-PAGE is performed on a cell lysate sample, and then proteins are transferred to nitrocellulose by electroblotting. Blotted nitrocellulose is blocked in freshly prepared PBS containing 3% nonfat dry milk for 20 min. at 25° C. Filters are incubated with the first antibody, and diluted in freshly prepared PBS-MILK at 4° C. overnight. Filters are washed twice with water and incubated in goat anti-mouse IgM linked to horseradish peroxidase, 1:1000 dilution (second antibody), in PBS-MILK for 1.5 h at room temperature. Filters are washed twice with water, then in PBS-0.05% Tween 20 for 3-5 min. Finally, filters are rinsed in 4-5 changes of water, and Ag-Ab complex products are visualized on the membrane using the ECL (Amersham) detection reagent kit.

b. Enzyme Linked Immunosorbent Assay (ELISA) Method

The levels of the molecules of interest are measured using total protein extracted from the above tissues. Total protein content is determined by a conventional protein assay (Bio-Rad). TGF-β1 (active and total) is determined using ELISA, as described in Roberts, A R, Wound Rep. Reg., 3, pages 408-418. The assay is performed before and after acidification of the tissue extracts that result in activation of latent TGF-βs, permitting the determination of the active (before acidification) and total TGF-β1. The activity of t-PA and PAI-1, and inactive complex between t-PA and PAI-1 (tPA/PAI-1 complex), are measured using commercially available ELISA kits for tPA and PAI-1 from American Diagnostica. The ELISA is based on the sandwich technique and includes the use of quenching antibodies to exclude falsely elevated results. To determine the levels of TGF-β1, tPA, PAI-1 and tPA/PAI-1 complex in tissue extracts, the samples are plotted against a standard curve. The protein content in tissue extracts is expressed as ng/mg of total protein. The standard curve is generated, and the tissue extract is diluted using the extraction buffer if required. The intra-assay coefficient of variation of the ELISA ranges from 5% to 8%, and the interassay coefficient variation ranges from 7-12%. MMP-1, TIMP-1, IL-1, FGF, and VEGF are assayed using commercially available ELISA kits, and used according to the given procedures.

c. Immunohistochemistry

Tissue sections prepared from the specimens are processed for localization of molecules as described, for example, in Ladin, D A et al., Wound Rep. Regen., 6, pages 28-37 (1998). Briefly, sections (5-6 μm thick) are cut and, following blocking of peroxidase activity with hydrogen peroxide and non-specific IgG binding site with corresponding normal serum, the sections are incubated with polyclonal goat antihuman and monoclonal antibodies (American Diagnostica). The concentration of antibodies used is 10 μg/ml of IgGs. Sections are washed and further processed by ABC Elit kits. Monoclonal antibodies to collagen types I and III and fibronectin at concentrations of 5 μg/ml of IgGs have been previously used with optimal results, and these same procedures are followed here. For immunolocalization of MMPs and TIMP-1, monoclonal antibodies to MMP-1 and TIMP-1 at concentrations of 2 and 10 μg/ml of IgGs have been previously used with optimal results. Deletion of primary antibodies, the use of primary antibodies preabsorbed with corresponding antigens during the staining procedures, and the replacement of primary antibodies with corresponding non-immune IgGs, are used as controls.

4. Measurement of Protein Activity:

a. Zymography Assay

Substrate Gel Zymography is used for functional evaluation of tPA activity, as previously described. Tissue extracts are mixed with an equal volume of 2× Laemmil sample buffer, and electrophoresed in 10% polyacrylamide slab mini-gels. Each experiment involves two gels, the first containing 0.1% gelatin to detect non plasminogen-dependent proteases, and the second containing gelatin and purified human plasminogen (Sigma) to detect plasminogen-dependent proteases. Molecular weight standards (Bethesda Research Laboratories, Bethesda, Md.) and recombinant tPA are applied to each gel. Following SDS-PAGE, gels are treated for 1 h with 2.5% Triton X-100 in 50 mM Tris-HCL, pH 8.0, at room temperature to remove the SDS. The gels are washed three times for 1 h with 50 mm Tris-HCL, pH 8.0, and then incubated for 16 h at 37° C., fixed, and stained with 0.1% Coomassie Brilliant Blue R250. Clearing of specific areas is compared densitometrically by the relative changes in the specific enzyme activity. The gel image is captured using a Kodak Digital camera and stored as TIFF files; the band density is determined and analyzed using NIH image analysis. This procedure allows identification by molecular weight of the active PA protein.

5. Results

The multiplex RT/PCR technique is used to quantitate mRNA levels in peritoneal tissues and cell culture for various key factors believed to contribute to the pathogenesis of adhesions. The advantage of the multiplex RT/PCR technique is that products are normalized to a housekeeping gene in the same tube. This normalization confers a number of benefits including standardization of 1) reverse transcription and PCR efficiencies, 2) pipetting differences between reactions, and 3) differences in template input.

To understand a possible paracrine and/or autocrine function that may take place in adhesion tissue, the co-localization of these molecules using the established in site hybridization and immunohistochemistry techniques is studied. The rationale for employing these techniques is to define the pathogenesis of adhesion development at the molecular level so that clinical care can be targeted to minimize such development and prevent its occurrence, thereby decreasing the morbidity of men and women with adhesions.

Explants with adhesions exhibit varying patterns of expression. Implants with adhesions have elevated mRNA and protein levels of TGF-β1, VEGF, and the extracellular matrix proteins (Type I collagen, Type III collagen, and fibronectin). Furthermore, plasminogen activator activity and matrix metalloproteinase function are suppressed, as evidenced by the reduction in levels of plasminogen activator and MMP-1 in conjunction with elevations in the levels of the inhibitors of these enzymes, PAI-I and TIMP-1 respectively. Alternatives for multiplex RT/PCR include G3PD or other housekeeping genes. Northern analysis is not an option because of sample size limitation.

Another technique which can be utilized is the RNase-protection assay. However, this methodology is time consuming and not as efficient as multiple RT/PCR. Differences identified in explants with and without adhesions can be used to minimize (and hopefully prevent) adhesion development. Elimination of adhesions would have a significant beneficial effect on women and men, by reducing adhesion related infertility, pelvic pain, and small bowel obstruction. Importantly, where possible, aliquots of specimens can be stored to allow future analysis of other moieties. These specimens can be available for future evaluations to determine the mechanism of the differential expression of molecules from explants with and without adhesions.

The regulation of adhesion development is multifaceted, involving genetic predisposition, type of injury, and extent of tissue damage (i.e., degree of initiation of tissue hypoxemia). Examination of explants from individuals undergoing intraabdominal surgery with and without extensive adhesions permits the investigation of the inter-individual variations in peritoneal healing which may help explain why different individuals form different amounts of adhesion in response to equivalent tissue trauma. Additionally, examination of explants from different sites/organs can be evaluated to define intra-individual variations in adhesion development at different sites throughout the abdominal cavity.

The adhesion model of this invention can be used to identify the existence of polymorphisms of key molecules in the healing process, such as plasminogen activator or TGF-β1, which might explain the apparent genetic predisposition to adhesion development. Additionally, the model can be used to identify time courses of the molecular changes provided for herein, as well as to identify other molecules known to be altered in explants, such as interleukins, rantes, integrins, and the IGF family, including evaluating the mechanisms responsible for changes in these molecules, such as intracellular signaling and the regulation of gene expression. Finally, this in vitro model of adhesion development can be utilized to screen the efficacy of adjuvants designed to reduce/prevent postoperative adhesions.

What is claimed is:

1. A biological model for adhesion development comprising a pair of opposed, viable explants of mammalian tissue maintained in culture under conditions sufficient to permit the formation of adhesions in vitro on one or both of the explant surfaces.

2. The biological model of claim 1 wherein the tissue is selected from the group consisting of peritoneum tissue, visceral tissue, subcutaneous tissue, and skin tissue.

3. The biological model of claim 2 wherein the tissue is peritoneum tissue.

4. The biological model of claim 1 wherein the mammal is a human.

5. The biological model of claim 1 in which the tissue is non-traumatized tissue.

6. The biological model of claim 1 in which the tissue is traumatized tissue.

7. The biological model of claim 1 wherein the culture media includes an oxygen concentration of from about 0% to about 21% oxygen by volume, preferably less than 5% oxygen by volume.

8. The biological model of claim 1 wherein the opposed tissue surfaces are spaced between about 1 mm and 20 mm apart, preferably between about 3 mm and 7 mm apart.

9. The biological model of claim 1 where a blood clot is formed in situ, or formed separately and placed between the opposed tissue surfaces.

10. The biological model of claim 1 wherein the culture media includes exogenous pro-fibrotic agents, preferably TGF-β1 or talc.

11. The biological model of claim 1 wherein the tissue explants are embedded in a material selected from the group consisting of collagen, synthetic extracellular matrix material and natural extracellular matrix material.

12. A method for forming an adhesion in vitro comprising the steps of
(a) preparing a biological model as described in claim 1 or claim 6,
(b) maintaining the explant tissues in culture for a period of from about 2 days to about 5 days, and
(c) allowing an adhesion to form on the tissue surface.

13. The method of claim 12 wherein the formation of the adhesion is regulated by the use of at least one substance added to the culture media.

14. A method for identifying a compound, molecule, biological substance, antibody, antisense DNA or device for preventing or remediating surgical adhesions comprising the steps of
(a) contacting the compound or device with the culture media containing the biological model of claim 1,
(b) evaluating whether, and to what extent, adhesions have formed at one or both of the tissue surfaces of the biological model after a defined period of time,
(c) comparing the extent of adhesion formation in step (b) with a control, or with the effect of other agents on the model, under substantially the same conditions, and
(d) observing whether the compound or device reduces the extent of adhesion formation.

15. A compound, molecule, biological substance, antibody, antisense DNA or device selected according to the method of claim 14.

16. An anti-adhesion pharmaceutical formulation which includes the compound, molecule, biological substance, antibody, antisense DNA or device of claim 15.

17. A method for treating a patient to prevent or reduce the extent of adhesion formation comprising administering to the patient the pharmaceutical formulation of claim 16.

18. A kit for e selecting optimal anti-adhesion agents for use in a patient to reduce adhesions comprising the biological model of claim 1, a tissue sample from the patient, and an anti-adhesion agent.

19. A kit for screening anti-adhesion drugs, devices or instruments for use in a patient comprising the biological model of claim 1, a tissue sample from the patient, and the drug, device or instrument.
